(19) United States
(12) Patent Application Publication
(10) Pub. No.: US 2025/0214955 A1
(43) Pub. Date: Jul. 3, 2025

(54) IMPROVED SYNTHETIC METHODS FOR MAKING LACTONES

(71) Applicant: P2 SCIENCE, INC., Woodbridge, CT (US)

(72) Inventors: Yong TU, Cheshire, CT (US); Yonghua YANG, East Lyme, CT (US); Patrick FOLEY, New Haven, CT (US); Ted ANASTASIOU, New Haven, CT (US); Tania SALAM, New Haven, CT (US); Alex MATTESON, Woodbridge, CT (US); Vivek BULBULE, Woodbridge, CT (US)

(21) Appl. No.: 18/867,252

(22) PCT Filed: May 19, 2023
(86) PCT No.: PCT/US23/67236

§ 371 Date: Nov. 19, 2024

Related U.S. Application Data
(60) Provisional application No. 63/343,919, filed on May 19, 2022.

(51) Int. Cl. C07D 307/33; C07D 307/83

(52) U.S. Cl. CPC C07D 307/33; C07D 307/83

(57) ABSTRACT

The present disclosure pertains to a new synthetic method for the preparation of lactones, an important class of organoleptic compounds which find use in the flavor and fragrance industries.

### CROSS-REFERENCE TO RELATED APPLICATIONS

This application is an international application which claims priority to U.S. Provisional Application Ser. No. 63/343,919, filed on May 19, 2022, the contents of which are hereby incorporated by reference in its entirety.

### FIELD OF INVENTION

The present disclosure pertains to a new synthetic method for the preparation of carboxylic acids, esters, and lactones, an important class of organoleptic compounds which find use in the flavor and fragrance industries.

### BACKGROUND

Lactones are very important molecules in the flavor and fragrance industry. For example, Mint lactone is a natural component of mint oil, and has been used as a precursor to 3,6-dimethylhexahydrobenzofuran-2-one (also known as Koumalactone®), a valued flavor and fragrance ingredient.

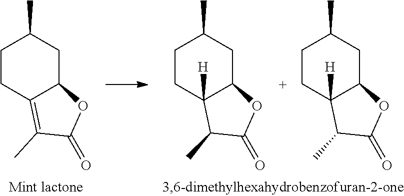

Multiple efforts have been made to cost effectively produce lactones such as mint lactone, and 3,6-dimethylhexahydrobenzofuran-2-one. Koch (U.S. Pat. No. 6,512,126) describe a hydrogenation and elimination of hydroxymenthofurolactone (I):

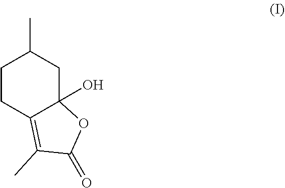

Xiong (CN 102,850,309) describes the treatment of 3-methylcyclohexanone (II), with methyl pyruvate in a multi-step synthesis involving sodium borohydride and iron chloride:

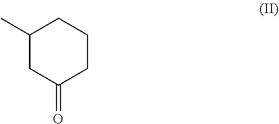

While both of these approaches are practicable, they use relatively expensive starting materials and reagents (e.g., Pd/C, NaBH₄). Another major drawback to these methods is that these methods do not produce highly enantio-enriched material. Additional approaches, including the use of citronellal (Shishido, et al., Tetrahedron Letters, 33(32), 4589-4592 (1992)), and alkynyl aldehydes (Gao et al., Journal of Organic Chemistry, 74(6), 2592 (2009)), have also been described, but again lack economic feasibility.

The naturally occurring, and commercially available, compound isopulegol (III), has been used as a key precursor in the synthesis of mint lactone and 3,6-dimethylhexahydrobenzofuran-2-one:

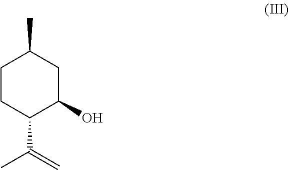

Although isopulegol (III) has been used as a starting material for the synthesis of enantiopure mint lactone in the past (Chavan et al., Tetrahedron Letters, 49(29), 6429-6436 (1993)), the approaches appear to have the common problem of being too costly to be commercially attractive, including the use of hydroboration and deprotonation with lithium diisopropylamide under cryogenic conditions.

As a result of these limitations, lactones, such as mint lactone, and 3,6-dimethylhexahydrobenzofuran-2-one, have been very expensive to obtain commercially, especially with the desired stereochemistry, and therefore their use has been limited.

It is even more difficult to selectively prepare asymmetric lactones having particular desired stereochemistries. As an example, Gaudin, Tetrahedron Letters 56(27), 4769-4776 (2000) and Gaudin, U.S. Pat. No. 5,464,824, report a route for making isomers of 3,6-dimethylhexahydrobenzofuran-2-one starting from isopulegol requiring double bond epoxidation, ring opening with lithium diisopropylamide to give isomeric allylic diols, followed by hydrogenation to form isomeric menthanediols, and then oxidation to form the lactone rings. Other similar routes are disclosed in Chinese patent application CN112010826A and U.S. Pat. No. 10,995,080. U.S. Pat. Nos. 10,399,954, and 11,008,299, also provide improved methods of making mint lactone and 3,6-dimethylhexahydrobenzofuran-2-one from isopulegol.

There remains a need for improved methods for the synthesis of lactones which rely on less hazardous, less costly and/or less toxic reagents, as well as a need for obtaining the highest yields using the least expensive starting materials. In view of these needs, a one-pot process for the synthesis of lactones from common starting materials would be particularly useful.

### BRIEF SUMMARY

The inventors have discovered a much-improved, economically feasible and relatively safe method for the synthesis of lactones, particularly in one pot from hydroxyalkenes. The present disclosure provides a method of making lactones in one pot according to the following scheme:

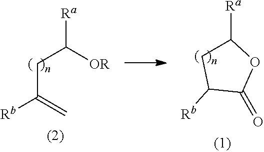

wherein R, R^{a}, R^{b} and n are as defined herein.

Without being bound by theory, it is believed the method proceeds by way of epoxidation of the double bond, rearrangement of the epoxide to an aldehyde, oxidation of the aldehyde to a carboxylic acid, and internal ring closure to form the lactone product.

### DETAILED DESCRIPTION

In a first aspect, the present disclosure therefore provides, a method (Method 1) of making a Compound of Formula I (Compound 1) comprising the step of treating a Compound of Formula II (Compound 2) with an oxidizing agent and an acid or base in a suitable solvent (e.g., an aqueous solvent):

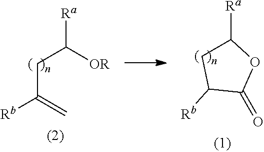

wherein the reaction proceeds in a single vessel without the isolation of any intermediates; wherein R is H or a protecting group (e.g., an ether, an ester, or a silyl ether protecting group), R^{a} and R^{b} are each independently selected from H, optionally substituted C₁₋₆alkyl, or optionally substituted aryl, or wherein R^{a} and R^{b} together form a 4-10 membered optionally substituted cycloalkyl ring, and n is an integer from 1 to 5 (e.g., 1, 2 or 3).

Without being bound by theory, it is believed that the one-pot procedure according to Method 1 comprises the intermediate steps of:
(a) epoxidizing the Compound (2) to form epoxide Intermediate Compound (3);
(b) rearranging epoxide Intermediate Compound (3) to form aldehyde Intermediate Compound (4);
(c) oxidizing aldehyde Intermediate Compound (4) to form carboxylic acid Intermediate Compound (5); and
(d) ring closing the carboxylic acid Intermediate Compound (5) to the Compound (1):

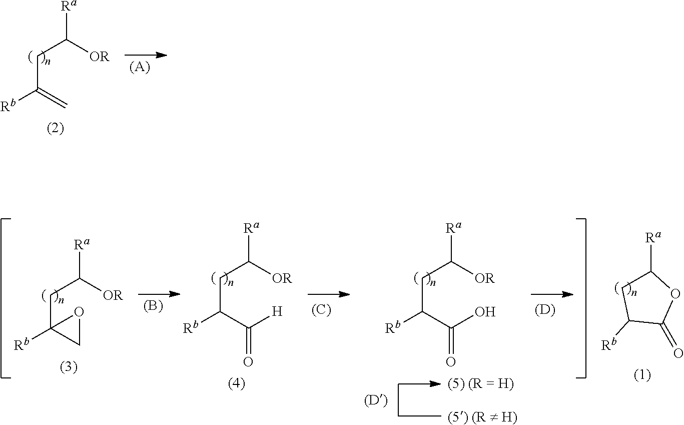

wherein R is H or a protecting group (e.g., an ether, an ester, or a silyl ether protecting group), R^{a} and R^{b} are each independently selected from H, optionally substituted C₁₋₆alkyl, or optionally substituted aryl, and n is an integer from 1 to 5 e.g., 1, 2 or 3). It is understood that if intermediate steps (A) through (D) occur, none of the Intermediate compounds are isolated and the reaction conditions of the Method provide for the occurrence of all necessary steps between Compound (2) and Compound (1).

In further embodiments of the first aspect, the present disclosure provides:
1.1 Method 1, wherein R is H;
1.2 Method 1, wherein R is an ester protecting group, e.g., R is —C(O)—R¹ or —S(O)₂—R¹, and wherein R¹ is H, C₁₋₆alkyl (e.g., methyl or ethyl), haloC₁₋₆alkyl (e.g., chloromethyl or trifluoromethyl), C₁₋₆alkoxy (e.g., methoxy or ethoxy), C₁₋₆alkoxymethyl (e.g., methoxyethyl or ethoxymethyl), aryl (e.g., phenyl or tolyl), arylmethyl (e.g., benzyl), aryloxy (e.g., phenoxy), or aryloxymethyl (e.g., phenoxymethyl);
1.3 Method 1.2, wherein R is —C(O)—R¹, and wherein R¹ is methyl, ethyl, propyl, isopropyl, or tert-butyl, or wherein R is —S(O)₂—R¹, and wherein R¹ is methyl;
1.4 Method 1.2, wherein R is —C(O)—R¹, and wherein R¹ is methyl;
1.5 Method 1.2, wherein R is —C(O)—R¹, and wherein R¹ is chloromethyl, dichloromethyl, trichloromethyl, 2,2,2-trichloroethyl, trifluoromethyl, methoxymethyl, phenoxymethyl, or benzyl, or wherein R is —S(O)₂—R¹, and wherein R¹ is trifluoromethyl, phenyl or tolyl;
1.6 Method 1, wherein R is an ether protecting group, e.g., R is unsubstituted C₁₋₆alkyl (e.g., methyl, ethyl, isopropyl, tert-butyl), or substituted C₁₋₆alkyl, such as C₁₋₆alkoxy-C₁₋₆alkyl, aryloxy-C₁₋₆alkyl, or aryl-C₁₋₆alkyl (e.g., —CH₂—O-Me, —CH₂—O—Et, —CH₂—S—Me, —CH₂—O—CH₂CH₂—OMe, CH₂—O—CH₂CCl₃, CH₂—O—CH₂CH₂—SiMe₃, —CH₂—O-Ph, —CH₂—O—CH₂-Ph, —CH₂—O—CH₂-(4-methoxyphenyl), —CH₂—O—CH₂-(3,4-dimethoxyphenyl), —CH₂CH₂—OEt, —CH₂CH₂Si(Me)₃, —CH₂CCl₃, —CH₂-Ph, —CH₂-(4-methoxyphenyl), —CH₂-(3,4-dimethoxyphenyl), —CH₂-(2,6-dimethoxyphenyl), or 2-tetrahydropyranyl;
1.7 Method 1, wherein R is a silyl ether protecting group, e.g., R is —Si(R²)(R³)(R⁴), wherein R², R³, and R⁴ are each independently selected from C₁₋₆alkyl (e.g., methyl, ethyl, isopropyl, tert-butyl, thexyl, benzyl), C₁₋₆alkoxy (e.g., methoxy, ethoxy, tert-butoxy), and aryl (e.g., phenyl);
1.8 Method 1.7, wherein R is selected from trimethylsilyl, triethylsilyl, triisopropylsilyl, dimethylisopropylsilyl, diethylisopropylsilyl, dimethylthexylsilyl, tert-butyldimethylsilyl, tert-butyldiphenylsilyl, tribenzylsilyl, triphenylsilyl, diphenylmethylsilyl, di-tert-butylmethylsilyl, tris(trimethylsilyl)silyl, tert-butylmethoxyphenylsilyl, and tert-butoxydiphenylsilyl;
1.9 Method 1 or any of 1.1 to 1.8, wherein the Intermediate Compound (3) is Compound (3a), Compound (3b), Compound (3c), Compound (3d), or a mixture thereof:

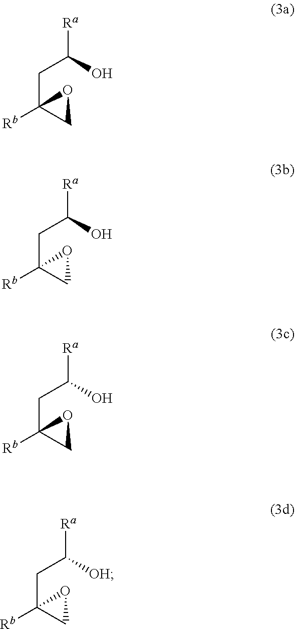

1.10 Method 1 or any of 1.1 to 1.9, wherein the Intermediate Compound (4) is Compound (4a), Compound (4b), Compound (4c), Compound (4d), or a mixture thereof:

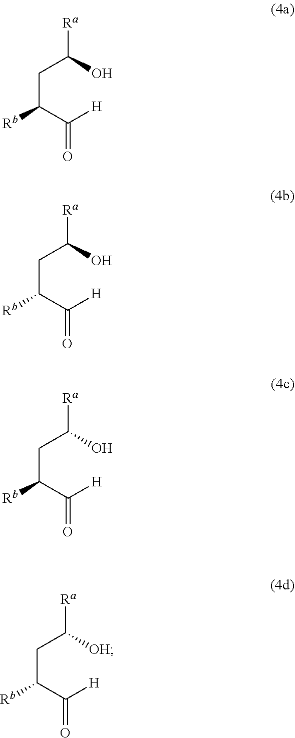

1.11 Method 1 or any of 1.1 to 1.10, wherein the Intermediate Compound (5) is Compound (5a), Compound (5b), Compound (5c), Compound (5d), or a mixture thereof:

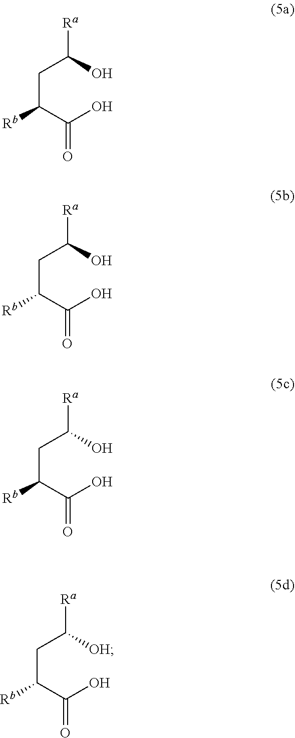

1.12 Method 1 or any of 1.1 to 1.11, wherein the Compound (1) is Compound (1a), Compound (1b), Compound (1c), Compound (1d), or a mixture thereof:

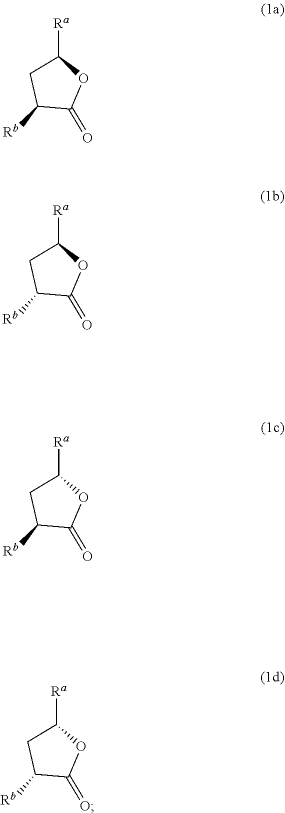

optionally, wherein the Compound (1) is enriched in one or more of the isomers (1a-1d), and optionally wherein the method further comprises the step of purifying or separating one or more of the isomers;
1.13 Method 1, or any of 1.1-1.12, wherein R^{a} and/or R^{b} is H;
1.14 Method 1, or any of 1.1-1.12, wherein R^{a} is optionally substituted C₁₋₆alkyl;
1.15 Method 1, or any of 1.1-1.12, wherein R^{a} is optionally substituted aryl;
1.16 Method 1, or any of 1.1-1.15, wherein R^{b} is optionally substituted C₁₋₆alkyl;
1.17 Method 1, or any of 1.1-1.15, wherein R^{b} is optionally substituted aryl;
1.18 Method 1, or any of 1.1-1.12, wherein R^{a} and R^{b} together form a 4-10 membered optionally substituted cycloalkyl ring (e.g., a cyclobutane, cyclopentane, or cyclohexane ring);
1.19 Method 1, or any of 1.1-1.18, wherein n is an integer selected from 1, 2, 3, 4 or 5;
1.20 Method 1, or any of 1.1-1.18, wherein n is an integer selected from 1, 2, and 3;
1.21 Method 1, or any of 1.1-1.18, wherein n is 1 or 2;
1.22 Method 1, or any of 1.1-1.21, wherein the oxidizing agent is selected from one or more of hydrogen peroxide, a chromium oxidant (e.g., chromium trioxide, chromic acid, pyridinium chlorochromate, potassium dichromate, chromium trioxide-pyridine complex, pyridinium dichromate), osmium tetroxide, potassium permanganate, peracetic acid, perchloric acid, perbenzoic acid, meta-chloroperoxybenzoic acid (mCPBA), trifluoroperacetic acid, periodic acid, magnesium monoperoxyphthalate, dimethyl dioxirane (DMDO), tert-butyl hydroperoxide, sodium hypochlorite, sodium tungstate, sodium periodate, potassium periodate, iodosyl benzene, pentafluoroiodosyl benzene, cumene hydroperoxide, potassium persulfate, potassium peroxymonosulfate, pyridine N-oxide, 2,6-dichloropyridine N-oxide, sodium chlorite, sodium hypochlorite, sodium chlorate, sodium perchlorate, or oxygen (e.g., in combination with a transitional metal catalyst, e.g., an iron catalyst), optionally in combination with any secondary reagents (e.g., secondary oxidants, catalysts, complexing agents, directing agents, reducing agent, or chiral auxiliaries);
1.23 Method 1.22, wherein the oxidizing agent is hydrogen peroxide, peracetic acid, trifluoroperacetic acid, meta-chloroperoxybenzoic acid, tert-butyl hydroperoxide, or potassium peroxymonosulfate;
1.24 Method 1, or any of 1.1-1.23, wherein the reaction is carried out using 1.0 to 5.0 equivalents of the oxidizing agent, e.g., 1.0 to 4.0 equivalents, or 1.0 to 3.0 equivalents, or 1.0 to 2.0 equivalents, or 1.5 to 4.0 equivalents, or 1.5 to 3.0 equivalents, or 1.5 to 2.5 equivalents, or 1.5 to 2.0 equivalents, or 2.0 to 4.0 equivalents, or 2.0 to 3.0 equivalents; and optionally 0.01 to 1.0 equivalents of any one or more additional reagents (e.g., secondary oxidant, or catalyst, or ligand, or other agent), e.g., 0.01 to 0.5 equivalents, 0.01 to 0.2 equivalents, or 0.01 to 0.1, or 0.01 to 0.05 equivalents;
1.25 Method 1, or any of 1.1-1.24, wherein the reaction comprises an acid catalyst;
1.26 Method 1.25, wherein the acid is a Bronsted acid, e.g., selected from hydrochloric acid, perchloric acid, peracetic acid, sulfuric acid, phosphoric acid, acetic acid, trifluoromethanesulfonic acid, methanesulfonic acid, trifluoroacetic acid, and nitric acid, or a heteropoly acid (e.g., phosphotungstic acid);
1.27 Method 1.26, wherein the acid is selected from sulfuric acid, phosphoric acid, trifluoromethanesulfonic acid, and nitric acid;
1.28 Any of Methods 1.25-1.27, wherein the reaction is carried out using 1.0 to 5.0 equivalents of the acid, e.g., 1.0 to 4.0 equivalents, or 1.0 to 3.0 equivalents, or 1.0 to 2.0 equivalents, or 1.5 to 4.0 equivalents, or 1.5 to 3.0 equivalents, or 1.5 to 2.5 equivalents, or 1.5 to 2.0 equivalents, or 2.0 to 4.0 equivalents, or 2.0 to 3.0 equivalents;
1.29 Method 1, or any of 1.1-1.24, wherein the reaction comprises a base catalyst;
1.30 Method 1.29, wherein the base is a Bronsted base, e.g., selected from an inorganic base (e.g., hydroxide, carbonate, or bicarbonate) or an organic base (e.g., an amine base);
1.31 Method 1.30, wherein the base is an inorganic base selected from lithium hydroxide, sodium hydroxide, potassium hydroxide, magnesium hydroxide, calcium hydroxide, lithium carbonate, sodium carbonate, potassium carbonate, lithium bicarbonate, sodium bicarbonate, and potassium bicarbonate;
1.32 Method 1.30, wherein the base is an amine base selected from triethylamine, diisopropylethylamine, N-methylpiperidine, N-methylmorpholine, pyridine, 4-dimethylaminopyridine, imidazole, N-methylimidazole, 1,5-diazabicyclo[4.3.0]non-5-ene (DBN), and 1,8-diazabicyclo[5.4.0]undec-7-ene (DBU),
1.33 Any of Methods 1.29-1.32, wherein the reaction is carried out using 1.0 to 5.0 equivalents of the base, e.g., 1.0 to 4.0 equivalents, or 1.0 to 3.0 equivalents, or 1.0 to 2.0 equivalents, or 1.5 to 4.0 equivalents, or 1.5 to 3.0 equivalents, or 1.5 to 2.5 equivalents, or 1.5 to 2.0 equivalents, or 2.0 to 4.0 equivalents, or 2.0 to 3.0 equivalents;
1.34 Method 1, or any of 1.1-1.33, wherein the reaction is carried out in a solvent selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid, sulfuric acid), acetic anhydride, carbon dioxide (e.g., supercritical carbon dioxide), and carbon disulfide, or a combination thereof;
1.35 Method 1.34, wherein the solvent is acetic acid and/or acetic anhydride and optionally wherein the reaction further comprises an acetate salt (e.g., sodium or potassium acetate);
1.36 Method 1, or any of 1.1-1.35, wherein the reaction comprises a polar protic solvent (e.g., water, an alcohol, an amine, or a thiol), in any amount (e.g., as solvent or in a catalytic amount, e.g., less than 0.5 molar equivalents);
1.37 Method 1.36, wherein the reaction comprises water (e.g., an aqueous solvent mixture);
1.38 Method 1.37, wherein any water present in the reaction is provided by the reagents (e.g., aqueous hydrogen peroxide, aqueous sulfuric acid, etc.);
1.39 Method 1, or any of 1.1-1.35, wherein the reaction is non-aqueous (e.g., no water is present), optionally wherein at least one non-aqueous polar protic solvent is present (e.g., an alcohol, amine or thiol);
1.40 Method 1, or any of 1.1-1.35, wherein the reaction comprises no polar protic solvents and/or wherein the reaction is non-aqueous (e.g., no water is present);
1.41 Method 1, or any of 1.1-1.40, wherein the reaction is carried out using a combination of hydrogen peroxide, sulfuric acid, and acetic acid, optionally 1.5-4 equivalents of each (e.g., 2-3 equivalents of each);
1.42 Method 1, or any of 1.1-1.40, wherein the reaction is carried out using a combination of acetic acid, peracetic acid, and sulfuric acid, optionally 1-3 equivalents of each (e.g., 1-1.5 equivalents of each);
1.43 Method 1, or any of 1.1-1.40, wherein the reaction is carried out using a combination of hydrogen peroxide, acetic acid, peracetic acid, and sulfuric acid, optionally 0.8-5 equivalents of each (e.g., 1-3 equivalents of each), optionally with sodium acetate (e.g., 0.05-0.24 equivalents);
1.44 Method 1, or any of 1.1-1.40, wherein the reaction is carried out using a combination of hydrogen peroxide, acetic anhydride, acetic acid, and sulfuric acid, optionally 0.8-5 equivalents of each (e.g., 1-3 equivalents of each), optionally with sodium acetate (e.g., 0.05-0.24 equivalents), further optionally with heptane co-solvent;
1.45 Method 1, or any of 1.1-1.40, wherein the reaction is carried out using a combination of potassium monoperoxysulfate, potassium hydrogen sulfate and potassium sulfate (e.g., Oxone®), in organic solvent (e.g., acetone/ethyl acetate), optionally 1-3 equivalents of potassium monoperoxysulfate (e.g., 1-2 equivalents);
1.46 Method 1, or any of 1.1-1.45, wherein the reaction is carried out using a combination of peracetic acid (e.g., 2-4 equiv. or about 3 equiv.), trifluoromethanesulfonic acid (e.g., 0.1-2 equiv., or about 0.5 equiv.), and acetic acid (e.g., 2-3 equiv., or about 2.5 equiv.);
1.47 Method 1, or any of 1.1-1.46, wherein the reaction is carried out at a temperature of 0° C. to 200° C., e.g., 0° C. to 150° C., or 0° C. to 100° C., or 0° C. to 75° C., or 0° C. to 50° C., or 0° C. to 25° C., or 25° C. to 200° C., or 25° C. to 150° C., or 25° C. to 100° C., or 25° C. to 75° C., or 25° C. to 50° C., or 25° C. to 30° C., or 50° C. to 200° C., or 50° C. to 150° C., or 50° C. to 100° C., or 75° C. to 200° C., or 75° C. to 150° C., or 75° C. to 100° C., or 80° C. to 150° C., or 80° C. to 100° C., or 90° C. to 150° C., or 90° C. to 125° C., or 90° C. to 110° C., or 90° C. to 100° C., or 90° C. to 95° C.;
1.48 Method 1, or any of 1.1-1.46, wherein the reaction is carried out at a temperature of 80° C. to 90° C., 20° C. to 70° C., 40° C. to 90° C., 20° C. to 50° C., or 20° C. to 30° C.;
1.49 Method 1, or any of 1.1-1.48, wherein the reactions steps (A), (B), (C), and/or (D) are carried out in a single vessel in a single reaction step, e.g., wherein step (B) spontaneously follows step (A), and step (C) spontaneously follow step (B), and step (D) spontaneously follows step (C);
1.50 Method 1, or any of 1.1-1.48, wherein the reactions steps (A), (B), (C), and/or (D) are carried out in a single vessel in a two reaction steps without isolation of any intermediates, e.g., wherein at least a second oxidizing agent is added in the second reaction step to initiate step (C), and wherein step (B) spontaneously follows step (A) and step (D) spontaneously follows step (C);
1.51 Method 1, or any of 1.1-1.50, wherein R is H, and wherein the reactant Compound (2) proceeds to the product Compound (1) in a single vessel, wherein the reaction is carried out by treating the Compound (2) with a combination of hydrogen peroxide, sulfuric acid, and acetic acid, optionally 1.5-4 equivalents of each (e.g., 2-3 equivalents of each, or 2 equivalents of each), optionally at a temperature of 75° C. to 150° C., or 80° C. to 100° C., or 90° C. to 95° C., or with a combination of peracetic acid (e.g., 2-4 equiv. or about 3 equiv.), trifluoromethanesulfonic acid (e.g., 0.1-2 equiv., or about 0.5 equiv.), and acetic acid (e.g., 2-3 equiv., or about 2.5 equiv.), optionally at a temperature of 25° C. to 50° C., or 25° C. to 30° C.;
1.52 Method 1, or any of 1.1-1.51, wherein the method further comprises a Step (2) of treating the Compound (1) with a base to cause enrichment of one or more isomers of Compound (1) by isomerization;
1.53 Method 1.52, wherein the base is selected from sodium hydride, potassium hydride, hydroxide bases (e.g., sodium hydroxide, potassium hydroxide), alkoxide bases (e.g., sodium tert-butoxide, potassium tert-butoxide, sodium methoxide, potassium methoxide, sodium ethoxide, potassium ethoxide), carbonate bases (e.g., sodium carbonate, potassium carbonate, cesium carbonate), bicarbonate bases (e.g., sodium bicarbonate), amide bases (e.g., lithium amide, lithium 2,2,6,6-tetramethylpiperide (LiTMP), lithium diisopropylamide (LDA), lithium bis(trimethylsilyl)amide) (LiHMDS)), alkyl lithium bases (e.g., sec-butyl lithium, tert-butyl lithium), and amine bases (e.g., triethylamine, diisopropylethylamine, N-methyl morpholine, DBU, DBN, pyridine, dimethylaminopyridine, imidazole);
1.54 Method 1.53, wherein the base is an alkoxide base (e.g., sodium tert-butoxide, potassium tert-butoxide, sodium methoxide, potassium methoxide, sodium ethoxide, potassium ethoxide) or an amide base (e.g., lithium amide, lithium 2,2,6,6-tetramethylpiperide (LiTMP), lithium diisopropylamide (LDA), lithium bis(trimethylsilyl)amide) (LiHMDS));
1.55 Method 1.54, wherein the base is sodium tert-butoxide or potassium tert-butoxide;
1.56 Any of Methods 1.52-1.55, wherein step (2) is carried out in a solvent selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), and ethers (e.g., diethyl ether, diisopropyl ether, methyl tert-butyl ether, tetrahydrofuran, 1,4-dioxane, dimethoxyethane, diethylene glycol dimethyl ether);
1.57 Method 1.56, wherein step (2) is carried out in a hydrocarbon solvent, e.g., pentane, hexane, heptane, or cyclohexane.
1.58 Any of Methods 1.52-1.57, wherein step (2) is carried out at a temperature of −78° C. to 200° C., e.g., −50° C. to 150° C., or −25° C. to 100° C., or 0 to 75° C., or 15° C. to 50° C., or 20° C. to 30° C., −78° C. to 0° C., or −50° C. to 0° C., or −25° C. to 25° C., or 25° C. to 75° C., or 50° C. to 100° C., or 75° C. to 150° C., or 100° C. to 150° C., or 125° C. to 200° C., or 150° C. to 200° C.;
1.59 Any of Methods 1.52-1.58, wherein Step (2) provides an enrichment in the amount of any one or more isomers, e.g., (1a), (1b), (1c), or (1d), of at least 10% by weight of the total weight of Compound (1), e.g., at least 15%, or at least 20%, or at least 25%;
1.60 Any of Methods 1.52-1.59, wherein Step (2) provides a product having at least 90% of any one isomer of Compound (1) or at least 90% of any pair of isomers of Compound (1);
1.61 Method 1, or any of 1.1-1.60, wherein the Method further comprises the crystallization of the product Compound (1) from a hydrocarbon solvent (e.g., pentane, hexane, heptane, cyclohexane, or a mixture thereof) at a temperature of less than 30° C., e.g., less than 25° C., or less than 15° C., or less than 10° C., or less than 5° C., or less than 0° C. to, or less than −5° C., or less than −10° C., or less than −20° C., or less than −30° C., or less than −40° C., or less than −50° C., e.g., as low as−78° C.;
1.62 Method 1.54, wherein the recrystallization step provides a product having at least 95%, or at least 96%, or at least 97%, or at least 98%, or at least 99% of any one isomer of Compound (1) or of any pair of isomers of Compound (1);
1.63 Any of Methods 1.52-1.62, wherein the Method comprises isomerization step (2) followed by recrystallization of the product (1);
1.64 Method 1.636, wherein the isomerization step (2) and the recrystallization step are performed as a continuous process in which each recrystallization is followed by an isomerization followed by a recrystallization, etc., until a desired purity of one or more isomers of Compound (1) is obtained;
1.65 Method 1, or any of 1.1-1.64, wherein the method further comprises a preliminary synthetic step (a protection step) of converting Compound (2′), wherein R is H, to the Compound (2), wherein R is not H (e.g., wherein R is a protecting group):

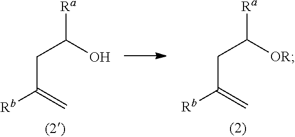

1.66 Method 1.65, wherein the protection step immediately precedes the step of treating the Compound (2) with an oxidizing agent, an acid or base, and a suitable aqueous solvent;
1.67 Method 1.65 or 1.66, wherein the protection step is carried out by treating the compound (2′) with a suitable protecting agent in a suitable solvent, optionally with a suitable base;
1.68 Method 1.67 wherein the protecting agent is selected from an acyl halide (e.g., acetyl chloride, benzoyl chloride, chloroacetyl chloride, dichloroacetyl chloride, trichloroacetyl chloride, methoxyacetyl chloride, phenoxyacetyl chloride, pivaloyl chloride, benzoyl chloride), an acyl anhydride (e.g., acetic anhydride, chloroacetic anhydride, dichloroacetic anhydride, trichloroacetic anhydride, trifluoroacetic anhydride, methoxyacetyl anhydride, phenoxyacetyl anhydride, pivaloyl anhydride, benzoyl anhydride), an alkyl halide (e.g., methoxymethyl chloride, methoxymethyl bromide, methoxyethyl chloride, methylthiomethyl iodide, benzyloxymethyl chloride, 4-methoxybenzyloxymethyl chloride, 2-methoxyethoxymethyl chloride, 2,2,2-trichloroethoxymethyl chloride, 2-trimethylsilylethoxymethyl chloride, 4-methoxybenzyl chloride, 4-methoxybenzyl bromide, 3,4-dimethoxybenzyl bromide,), a silyl reagent (e.g., the chloride, silane or triflate of the group trimethylsilyl, triethylsilyl, triisopropylsilyl, dimethylisopropylsilyl, diethylisopropylsilyl, dimethylthexylsilyl, tert-butyldimethylsilyl, tert-butyldiphenylsilyl, tribenzylsilyl, triphenylsilyl, diphenylmethylsilyl, di-tert-butylmethylsilyl, tris(trimethylsilyl)silyl, tert-butylmethoxyphenylsilyl, and tert-butoxydiphenylsilyl), or other protecting agents (e.g., formic acid, acetic acid, ethyl formate, methyl formate, chloroacetic acid, dihydropyran, 2-hydroxytetrahydropyran, ethyl vinyl ether, trimethylsilylethoxyethene, isobutylene, methanesulfonyl chloride, trifluoromethanesulfonyl chloride, trifluoromethanesulfonic anhydride, N,N-bis(trifluoromethanesulfonyl)aniline, benzenesulfonyl chloride, toluenesulfonyl chloride);
1.69 Method 1.67 or 1.68, wherein the suitable base is selected from sodium hydride, potassium hydride, hydroxide bases (e.g., sodium hydroxide, potassium hydroxide), alkoxide bases (e.g., sodium tert-butoxide, potassium tert-butoxide), carbonate bases (e.g., sodium carbonate, potassium carbonate, cesium carbonate), bicarbonate bases (e.g., sodium bicarbonate), and amine bases (e.g., triethylamine, diisopropylethylamine, N-methyl morpholine, DBU, DBN, pyridine, dimethylaminopyridine, imidazole);
1.70 Any of methods 1.67-1.69, wherein the suitable solvent is selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid), carbon dioxide (e.g., supercritical carbon dioxide), and carbon disulfide, or combinations thereof, or wherein the solvent is a neat reagent (e.g., pyridine);
1.71 Any of Methods 1.67-1.70, wherein the protection step is carried out using 1.0 to 5.0 equivalents of the protecting agent, e.g., 1.0 to 4.0 equivalents, or 1.0 to 3.0 equivalents, or 1.0 to 2.0 equivalents, or 1.0 to 1.50 equivalents, or 1.0 to 1.25 equivalents, or 1.0 to 1.15 equivalents, or 1.0 to 1.05 equivalents, or 1.5 to 2.0 equivalents, or 2.0 to 3.0 equivalents;
1.72 Any of Methods 1.67-1.71, wherein the protection step is carried out at a temperature of −100° C. to 200° C., e.g., −50° C. to 150° C., or −100° C. to 0° C., or −100° C. to −50° C., or −50° C. to 0° C., or −25° C. to 0° C., or −25° C. to 25° C., or 0° C. to 200° C., or 0° C. to 150° C., or 0° C. to 100° C., or 0° C. to 50° C., or 0° C. to 25° C., or 25° C. to 150° C., or 25° C. to 100° C., or 50° C. to 200° C., or 50° C. to 100° C., or 75° C. to 200° C., or 100° C. to 200° C., or 150° C. to 200° C.;
1.73 Method 1, or any of 1.1-1.72, wherein the method proceeds through a further intermediate step (D′) of converting Intermediate Compound (5′), wherein R is not H (e.g., wherein R is a protecting group), to Intermediate Compound (5″), wherein R is H:

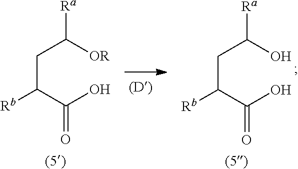

wherein the protecting group R is spontaneously eliminated during method (i.e., no synthetic deprotection step D′ is needed);
1.74 Method 1, or any of 1.1-1.73, wherein the method does not comprise any synthetic steps other than the step of treating the Compound (2) with an oxidizing agent, an acid or base, and a suitable solvent or solvents, and optionally the protection step, one or more isomerization steps (2), and/or one or more crystallization steps;
1.75 Method 1, or any of 1.1-1.74, wherein the method does not comprise the use of any reagents or reactants other than the Compound (2), and the reagents set forth herein (e.g., acids, bases, oxidizing agents, catalysts, protecting agents, deprotecting agents, solvents), for example, the method does not comprise the use of any carbon monoxide, carbonyl equivalents, or enzymes).

It is understood that in reference to “synthetic steps” the present disclosure recites specific chemical transformations which may optionally be accompanied by various procedural steps known to those skilled in the art, such as the stepwise addition of reagents, heating steps, cooling steps, quenching steps, precipitation steps, mixing steps, drying steps, evaporation steps, and other steps of purification (e.g., aqueous extraction, chromatography, distillation) and steps of analysis (e.g., thin-layer chromatography, MS, LCMS, NMR, elemental analysis, etc.). In embodiments wherein the invention of the present disclosure is limited to specified steps, it is understood that such limitation applies to the synthetic steps carried out and no limitation is made on the procedural steps involved in carrying out the method except where specifically indicated. The term “reaction steps” refers to the number of procedural steps carried out involving the addition of distinct selections of reagents.

In some embodiments, the mechanistic steps (A), (B), (C), and (D) may be combined into only two reaction steps. Without being bound by theory, it is believed that this may be accomplished because step (B) can occur spontaneously under thermal or acid-catalyzed conditions promoted by the reaction conditions of step (A), and likewise, step (D) can occur spontaneously under thermal or acid-catalyzed conditions promoted by the reaction conditions of step (C). Thus, a two-step reaction sequence may be employed in which oxidizing agent, optionally acid, and solvent are added in a first reaction step (e.g., hydrogen peroxide or peracetic acid, in sulfuric acid and/or acetic acid and/or acetic anhydride), followed after a period of time by a second oxidizing agent, an acid and solvent (e.g., hydrogen peroxide or peracetic acid, in sulfuric acid and/or acetic acid and/or acetic anhydride). This two-step sequence may be carried out in two reaction vessels or in a single reaction vessel (i.e., sequential addition of the two sets of reagents separated in time). When the two-step sequence is used, either the reaction mixture of the first step can be added to the reagents and solvents for the second step, or vice versa.

It is further understood that references to “intermediate” compounds and “intermediate steps” refers to non-synthetic interventions which occur during the synthetic steps recited herein. Thus, no further manipulation is necessary for the intermediate compounds to form or for the intermediate steps to occur. Information pertaining to intermediate compounds and intermediate steps is provided herein merely for example of how the mechanism of the claimed reaction may occur.

The present invention is based on the unexpected discovery that a single vessel (“one-pot”) procedure can be used to convert the Compound (2) to the Compound (1), by treating the Compound (2) with an oxidizing agent, and an acid, in a suitable solvent. In a preferred embodiment, the oxidizing agent is hydrogen peroxide, acid is sulfuric acid (e.g., 60% aq.) and the solvent is acetic acid. Without being bound by theory, it is believed that the hydrogen peroxide and the acetic acid solvent may form peracetic acid in-situ. In another preferred embodiment, the reaction may be conducted using peracetic acid, trifluoromethanesulfonic acid, and acetic acid. In another preferred embodiment, the oxidizing agent is peracetic acid, the acid is sulfuric acid (e.g., 60% aq.) and the solvent is acetic acid and/or acetic anhydride. In some embodiments an acetate salt is added adjust the acidity of the reaction or to provide a buffering effect, for example, sodium acetate or potassium acetate.

In some embodiments, the reactions described in the present disclosure may also proceed via various other intermediates. For example (i.e., wherein n=1, R=H):

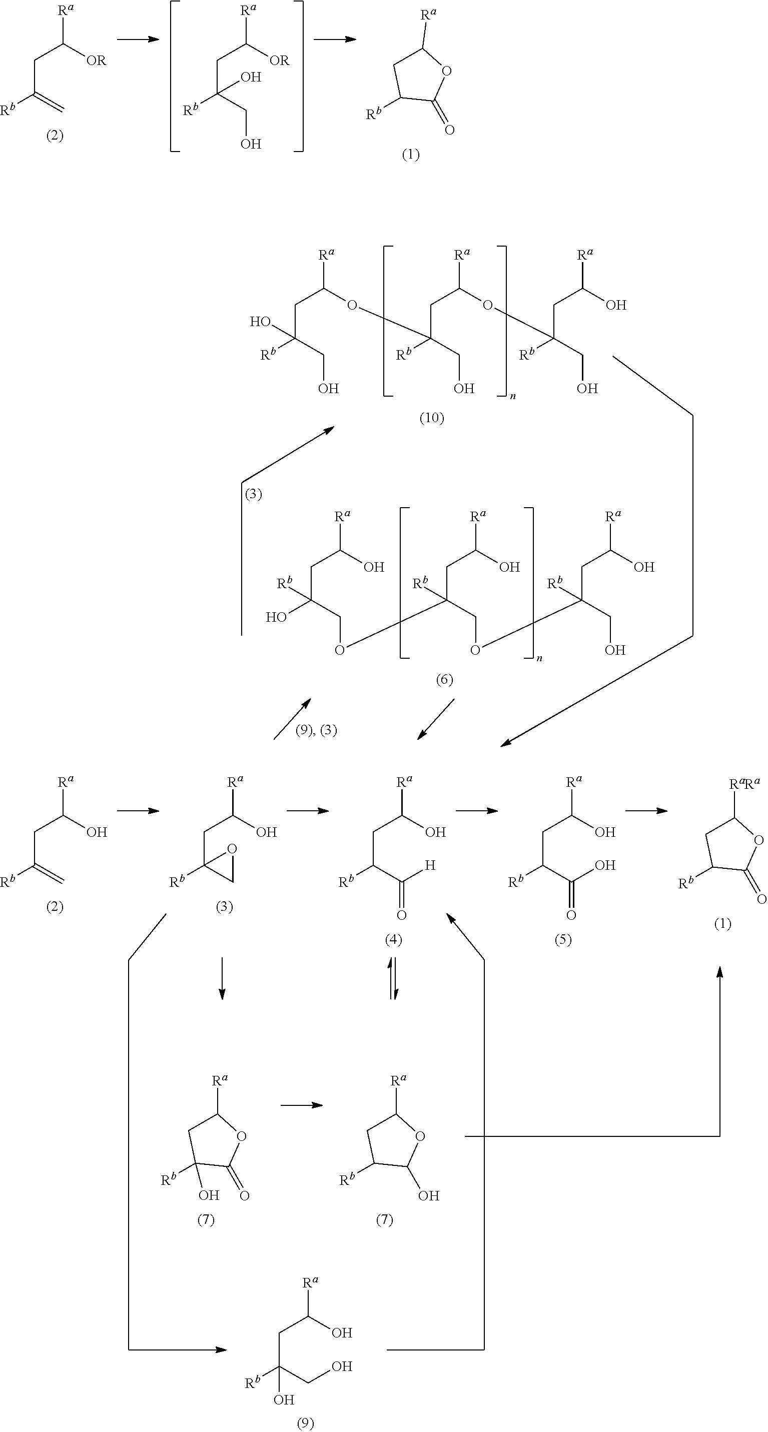

As shown above, under the one-pot reaction conditions described herein (e.g., hydrogen peroxide, sulfuric acid, acetic acid), the initially formed epoxide (3) may undergo an acid-catalyzed polymerization with intermediate (9) to form short oligomers (6), which may undergo oxidative depolymerization to form aldehydes (4). In addition, the initially formed epoxide (3) may undergo an acid-catalyzed self-polymerization to form short oligomers (10), which may also undergo oxidative depolymerization to form aldehydes (4) In addition, the epoxide (3) may undergo an acid-catalyzed intramolecular ring closure to form tertiary alcohol (7) which may rearrange to cyclic hemiacetal (8). This hemiacetal is reversibly formed by intramolecular condensation of aldehyde (4), or may be oxidized directly to the lactone product (1). In addition, epoxide (3) may undergo acid-catalyzed hydrolysis to form vicinal diol (9), which may undergo acid-catalyzed elimination to form aldehyde (4) (initially as its enol tautomer). While many of these individual steps are indicated as being acid-catalyzed, without being bound by theory, it is believed that many of them may also take place under base-catalyzed conditions. Thus, without being bound by theory, it is believed that mechanisms taking place in the one-pot procedure may be catalyzed by acid or base, or may be autocatalytic, in a polar medium (e.g., a medium comprising at least one polar solvent, e.g., water, an alcohol, an amine, a thiol, or other polar protic or polar aprotic solvents).

Certain isomers of the Compound (1) described in the present disclosure may be preferred commercially due to their more favorable olfactory effects compared to alternative isomers (e.g., of Compounds (1a), (1b), (1c), or (1d). Advantageously, Method 1 et seq. and provide an improvement over the prior art in that the methods tend to provide an excess of one or more of the intermediate isomers (1a), (1b), (1c), or (1d) (i.e., a racemic product is not usually obtained). In some embodiments of the present disclosure, the preference for one or more of said isomers may be further enhanced by subjecting the initial products to a base-catalyzed isomerization reaction, such as, by treating the initial product (1) with sodium tert-butoxide in a hydrocarbon solvent, or similar methods. This can substantially increase the amounts of particular isomers produced by the method.

In some embodiments of the present disclosure, the preference for one or more of the isomers of Compound (1) may be further enhanced by subjecting the initial products to a crystallization procedure which selectively crystalizes one or more of said isomers. For example, the initial product (1) may be crystallized from a hydrocarbon solvent at a temperature under 0° C. Optionally, by performing the Method 1 with a base-catalyzed isomerization step and a crystallization step, product (1) can be obtained having 99% or more of one isomer or one pair of isomers.

In some embodiments, the Compound (1) is a bicyclic compound because the substituent groups R^{a} and R^{b} form a carbocyclic ring. For example, R^{a} and R^{b} can form a 3-carbon bridge, 4-carbon bridge, or 5-carbon bridge, resulting in, respectively, a bicyclic structure with a cyclopentane, cyclohexane, or cycloheptane ring:

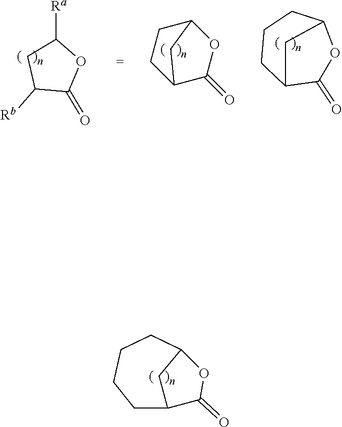

Said bridge formed by the R^{a} and R^{b} substituent groups may be optionally substituted as set forth herein.

As used herein, optionally substituted means that the indicated functional group is either unsubstituted or substituted by one or more groups up to the maximum permitted by the rules of valency wherein said groups are selected from: halo, hydroxy, cyano, C₁₋₆alkyl, C₂₋₆alkenyl, C₂₋₆alkynyl, C₃₋₆cycloalkyl, C₁₋₆haloalkyl, —O—Si(R^{x})₃, —O—R^{x}, —C(O)H, —C(O)—R^{x}, —C(O)—O—R^{x}, —C(O)—NH—R^{x}, —C(O)—N—(R^{x})(R^{x}), —O—C(O)—R^{x}, —NH(R^{x})—C(O)—R^{x}, —N(R^{x})(R^{x})—C(O)—R^{x}), —NH(R^{x}), —N(R^{x})(R^{x}), aryl, and heteroaryl; wherein each of said C₁₋₆alkyl, C₃₋₆cycloalkyl, aryl or heteroaryl is further optionally substituted by one or more halo, hydroxy, cyano, C₁₋₆alkyl, C₂₋₆alkenyl, C₂₋₆alkynyl, C₃₋₆cycloalkyl, C₁₋₆haloalkyl, —O—Si(R^{x})₃, —O—R^{x}, —C(O)H, —C(O)—R^{x}, —C(O)—O—R^{x}, —C(O)—NH—R^{x}, —C(O)—N—(R^{x})(R^{x}), —O—C(O)—R^{x}, —NH(R^{x})—C(O)—R^{x}, —N(R^{x})(R^{x})—C(O)—R^{x}), —NH(R^{x}), —N(R^{x})(R^{x}), aryl, and heteroaryl; and wherein each R^{x} is independently selected from hydrogen, C₁₋₆alkyl, C₂₋₆alkenyl, C₂₋₆alkynyl, C₃₋₆cycloalkyl, aryl and heteroaryl. Aryl includes, but is not limited to, optionally substituted phenyl, and optionally substituted naphthyl. Heteroaryl includes, but is not limited to, any optionally substituted furan, thiophene, pyrrole, pyrazole, oxazole, isoxazole, thiazole, isothiazole, pyridine, pyrimidine, pyridazine, pyrazine, indole, benzofuran, benzoxazole, benzisoxazole, benzothiazole, quinoline, isoquinoline, quinazoline, and quinoxaline.

As used herein, the term “one-pot” refers to a process which can be involve one reaction step, two reaction steps, or more reaction steps, as long as all reaction components are present together (aside from when reagents are being added) and no purification step is performed between the reaction steps (e.g., no aqueous work-up, extraction, filtration, precipitation, chromatography, or distillation, or any other procedures which would remove any components from the reaction mixture). A one-pot procedure can involve adding all reagents at the outset of the process and allowing all necessary mechanistic steps to take place through completion of the process (i.e., a one-reaction step process), or the one-pot procedure can involve adding one set of reagents at the outset and a different set of reagents later, whereby it is expected that only some of the mechanistic steps take place after the first reagent addition(s) and the remaining reaction steps take place after the final reagent addition(s).

Therefore, as used herein, the term “one-pot” refers to the fact that multiple mechanistic or reaction steps occur without purification, not necessarily that only a single reaction vessel is involved, although this may be the case. For example, where the “one-pot” process involves two reaction steps, it may be necessary to involve a second reaction vessel. Thus, in the two-reaction step process, the reagents for the second reaction step may be added directly to the reaction mixture from the first reaction step. In such case, only a single reaction vessel is necessary. However, alternatively, the reaction mixture from the first reaction step may be added directly to the reagents for the second reaction step, which are then necessarily in a second vessel initially. But upon completion of this addition, the reaction again involves a single vessel holding all of the reagents, intermediates, reactants, and products from all steps, thus making this still a “one-pot” procedure.

In a second aspect, the present disclosure provides Compound (1) made according to Method 1 or any of 1.1 et seq.

In a third aspect, the present disclosure provides a composition, such as an organoleptic composition, comprising Compound (1), made according to Method 1 or any of 1.1 et seq., wherein said compound imparts a flavor or fragrance to the composition. In some embodiments, the composition is a flavor or fragrance composition, for example, further comprising one or more additional flavor or fragrance agents or additives, and at least one solvent, or carrier. The composition may be a liquid or solid composition, such as a soft or waxy solid. The composition may further comprise one or more excipients, such as polymers, gelling agents, powdery substrates, surfactants, emollients, plasticizers, wetting agents, swelling agents, or active agents (e.g., an oral care active or a medicinal active agent), or any other cosmetically acceptable or orally acceptable additives. In some embodiments said compound, or said composition, is used to impart a flavor or fragrance to a product, such as a consumer product.

Suitable solvents for said compositions include: water, methanol, ethanol, propanol, isopropanol, butanol, 3-methoxy-3-methyl-1-butanol, benzyl alcohol, ethyl carbitol (diethylene glycol monoethyl ether), dimethyl ether, diethyl ether, diisopropyl ether, methyl tert-butyl ether, ethylene glycol, propylene glycol, dipropylene glycol, butylene glycol, hexylene glycol, glycerin, ethylene glycol monomethyl ether, propylene glycol monomethyl ether, propylene glycol monoethyl ether, dipropylene glycol monomethyl ether, isoparaffin, paraffin, limonene, pinene, triethyl citrate, triacetin, benzyl benzoate, isopropyl myristate, triglycerides, liquid waxes, propylene glycol derivatives, ethylene glycol derivatives, other alcohols or ethers, or any combination thereof.

As used herein, the term “aqueous solvent” refers to a solvent mixture having any amount of water, including trace water (for example, where the solvent is one that is miscible with water). The water for the “aqueous solvent” may also be provided by aqueous reagents (such as aqueous acids, e.g., sulfuric acid, or aqueous oxidizing agents, such as hydrogen peroxide). Without being bound by theory, it is believed that water is only necessary in a catalytic amount for the methods described herein to proceed, so significant amounts of water need not be used in the reactions.

It is understood that hydrogen peroxide and peracetic acid (also known as peroxyacetic acid) are both commonly available as solutions, not as pure compounds. Hydrogen peroxide is sold as an aqueous solution consisting essentially of 10-70% hydrogen peroxide and the balance water, but with small amounts of other ingredients (e.g., <5% of stabilizing agents or impurities). Peracetic acid is commercially made by oxidizing dilute acetic acid with hydrogen peroxide. As a result, peracetic acid is commonly sold as a solution of 15% or 32% peracetic acid in an aqueous acetic acid carrier. The solution will commonly have small amounts of unreacted hydrogen peroxide, as well as possibly stabilizing agents or other impurities.

It is further understood that the oxidizing agent potassium monoperoxysulfate (also known as “MPS”, having the formula KHSO₅) is commonly sold under the trade names Oxone® and Caroat®, which are triple salt mixtures consisting of about 2 parts KHSO₅, 1 part KHSO₄, and 1 part K₂SO₄, and being approximately 47-50% by weight of KHSO₅. The active oxygen species KHSO₅ is more stable in this mixture than in pure form.

As used herein, the term “fragrance composition” means a mixture of fragrance ingredients, including auxiliary substances if desired, dissolved in a suitable solvent or mixed with a powdery substrate used to provide a desired odor to a product. Examples of products having fragrance compositions include, but are not limited to, perfumes, soaps, insect repellants and insecticides, detergents, household cleaning agents, air fresheners, room sprays, pomanders, candles, cosmetics, toilet waters, pre-and aftershave lotions, talcum powders, hair-care products, body deodorants, anti-perspirants, and pet litter.

As used herein, the term “flavor composition” means a mixture of flavor ingredients, including auxiliary substances if desired, dissolved in a suitable solvent or mixed with a powdery substrate used to provide a desired flavor to a product. Examples of products having flavor compositions include, but are not limited to, dental hygiene products such as mouth wash, toothpaste, floss, and breath fresheners, orally administered medications including liquids, tablets or capsules, and food products.

Fragrance and flavor ingredients and mixtures of fragrance and flavor ingredients that may be used in combination with the Compound (1), for the manufacture of fragrance and flavor compositions include, but are not limited to, natural products including extracts, animal products and essential oils, absolutes, resinoids, resins, and concretes, and synthetic fragrance materials which include, but are not limited to, alcohols, aldehydes, ketones, ethers, acids, esters, acetals, phenols, ethers, lactones, furansketals, nitriles, acids, and hydrocarbons, including both saturated and unsaturated compounds and aliphatic carbocyclic and heterocyclic compounds, and animal products.

The present disclosure further provides for the Compound (1), made according to Method 1 or any of 1.1 et seq., as a flavor or fragrance agent, e.g., in order to impart a flavor or fragrance to a composition or product.

In a fourth aspect, the present disclosure provides a product or composition, such as an organoleptic composition, comprising Compound (1), made according to Method 1 or any of 1.1 et seq. In some embodiments, the Compound (1) may be used alone as a fragrance or added into a fragrance composition and/or consumer product as an agent for increasing substantivity and/or retention of a fragrance preparation and/or as a fixative.

Suitable solvents may include, alcohols such as methanol, ethanol, propanol, isopropanol, butanol, tert-butanol and the like, lower alkyl ester of lower carboxylic acid such ethyl acetate and the like; alkane nitriles such as acetonitrile, propionitrile, butyronitrile, isobutyronitrile, valeronitrile and the like; aromatic hydrocarbons such as benzene, toluene, xylene, anisole and the like; aliphatic hydrocarbons such as pentane, hexane, heptane, octane, cyclohexane, cycloheptane, cyclooctane and the like; and water. All these solvents can be used singly or in mixture with each other. Water can also be used as a solvent with or without mixing above mentioned solvents during the reaction.

Without being bound by theory, it is believed that Method 1 et seq. begins with epoxidation of the double bond, followed by a later oxidation of an aldehyde intermediate to form a carboxylic acid intermediate. Therefore, it is believed that the method may be carried out using any oxidizing agent capable of both epoxidation and aldehyde oxidation. Epoxidation is often carried out using a strong oxidizing agent, such as mCPBA, or a combination of oxygen with a catalyst, or using a combination of oxidizing agents of differing strengths. Commonly used reagents include one or more of hydrogen peroxide, osmium tetroxide, peracetic acid, perchloric acid, perbenzoic acid, meta-chloroperoxybenzoic acid, trifluoroperacetic acid, magnesium monoperoxyphthalate, dimethyl dioxirane (DMDO), tert-butyl hydroperoxide, sodium hypochlorite, sodium tungstate, or oxygen (e.g., in combination with a transitional metal catalyst, e.g., an iron catalyst). In some embodiments, transition metal-doped silica or zeolite catalysts may be used with oxygen and a secondary oxidant, such as tert-butyl hydroperoxide, or with a co-reagent, such as an aliphatic aldehyde. For example, epoxidation can be carried out according to the procedure of Madadi et al., Applied Cat. B: Environ. 260 (2020) 118049, using the catalyst mesoporous SBA-16 silica modified with cobalt, titanium, nickel, iron, or manganese, and reacted with the alkene substrate in the presence of oxygen and a C₂₋₁₀ aliphatic aldehyde (e.g., isobutyraldehyde). In some embodiments, oxygen or peroxides (e.g., hydrogen peroxide) can be used as the oxidant in combination with a C₂₋₁₀ aliphatic aldehyde (e.g., isobutyraldehyde), and a transition metal porphyrin complex (e.g., tetraphenyl porphyrin), as described in Chinese patent publication CN1915983A (“Method for preparing epoxy compound by oxidizing olefin or cycloolefin through bionic catalytic oxygen”). Suitable transition metals for the catalyst include manganese, iron, or ruthenium, and the phenyl groups of the porphyrin ring may be unsubstituted or para-and/or ortho-substituted with electron withdrawing groups (e.g., nitro, fluoro, chloro). Other suitable transition metals include titanium, chromium, molybdenum, osmium, and cobalt, while other suitable oxidants include iodosyl benzene, pentafluoroiodosyl benzene, mCPBA, sodium hypochlorite, tert-butyl hydroperoxide, cumene hydroperoxide, potassium persulfate, pyridine N-oxide, and 2,6-dichloropyridine N-oxide. See Amal Salmeen Basaleh, “The Kinetics and Mechanism of the Activation of Metalloporphyrin by Hydrogen Peroxide,” Dissertation (Univ. of Surrey, Guildford, U.K, & King Abdulaziz. Univ., Jedda, Saudi Arabia) (July 2013) A particularly effective epoxidation method employs hydrogen peroxide with catalytic sodium tungstate, and optionally various additives, such as sodium sulfate, methyl-tri-n-octylammonium hydrogen sulfate, and/or phenylphosphonic acid (Noyori oxidation), such as reported by Hachiya et al., Syn. Lett. 19:2819-22 (2011). A simple mCPBA-mediated epoxidation of isopulegol is reported by Zhao et al., Tet. Lett. 45(19): 2713-16 (2004).

In some embodiments wherein R is H, use can be made of the hydroxy group for a directed (e.g., asymmetric) epoxidation under mild conditions. For example, as described in Gill et al., Chem. Commun. 1743-1744 (1996), the epoxidation may be carried out using vanadyl acetylacetonate (VO(acac)₂), in the presence of tert-butyl hydroperoxide in benzene solvent (or toluene). Guidotti et al., Chem. Commun. 1789-1790 (2000) similarly report the epoxidation of isopulegol using mesoporous titanium MCM-41 complex with tert-butyl hydroperoxide. The use of the simple oxidation system Oxone/acetone/sodium bicarbonate has been reported by Ferraz et al, Tet. Lett. 41(26): 5021-23 (2000).

Typically, an aldehyde to carboxylic acid oxidation is carried out using a strong oxidizing agent, such as a chromium oxidant (e.g., chromium trioxide, chromic acid, pyridinium chlorochromate, potassium dichromate, chromium trioxide-pyridine complex, pyridinium dichromate), osmium tetroxide, potassium permanganate, silver oxide, hydrogen peroxide, peracetic acid, perchloric acid, trifluoroperacetic acid, periodic acid, potassium periodate, sodium chlorite, oxygen and N-hydroxyphthalimide, or potassium monoperoxysulfate. Jones Reagent (chromium trioxide in aqueous sulfuric acid) is a particularly effective oxidizing system. Chinese patent application CN108315499A also discloses an environmentally friendly oxidation method using an oxygen atmosphere with an N-hydroxyimide catalyst, such as N-hydroxyphthalimide, N-hydroxymaleimide, N-hydroxysuccinimide, N-hydroxyglutarimide N-hydroxy-1,8-napthalimide, or N-hydroxy-benzene dicarboximide (e.g., 0.05-0.15 equivalents, in a suitable solvent, e.g., acetonitrile), optionally in conjunction with a nitrite agent (e.g., methyl nitrite, ethyl nitrite, propyl nitrite, isopropyl nitrite, butyl nitrite, isoamyl nitrite, tert-butyl nitrite, or benzyl nitrite).

Without being bound by theory, it is believed that Method 1 et seq. proceeds via a rearrangement step which converts the initially formed epoxide into an aldehyde. Therefore, it is believed that the method may be carried out using any acid capable of catalyzing such a rearrangement. Such rearrangement has been reporting using such acids as 1% sulfuric acid in glacial acetic acid, p-toluenesulfonic acid, magnesium bromide diethyl etherate, zinc bromide, zinc chloride, lithium bromide, boron trifluoride, and borane. See Arata & Tanabe, Catal. Rev. Sci. Eng. 25(3): 365-320 (1983). Another potential acidic catalyst for this rearrangement is the heteropolyacids, such as phosphotungstic acid (H₃PW₁₂O₄₀), such as similarly described in Gusevskaya et al., Chem. Eur. J. 14:6166-72 (2008). Stork et al., JACS 118(43): 10660-61, report a typical set of conditions for epoxidation of an exocyclic double bond using mCPBA followed by rearrangement of the epoxide to the aldehyde catalyzed by boron trifluoride etherate complex.

### EXAMPLES

NMR spectra are recorded using a 500 MHz NMR spectrometer. All ¹H-NMR data are reported in δ units, parts per million (ppm), and are calibrated relative to the signals for residual chloroform (7.26 ppm) in deuterochloroform (CDCl₃). All ¹³C-NMR data are reported in ppm relative to CDCl₃ (77.16 ppm) and are obtained with 1H decoupling. The following abbreviations or combinations thereof were used to explain the multiplicities: s=singlet, d=doublet, t=triplet, q=quartet, br=broad, m=multiplet, a=apparent.

GC Analysis is performed on an Agilent 6890N gas chromatograph with a Restek-Stabilwax (crossbond Carbowax Polyethylene glycol) 30 mm×0.25 mm×0.25 μm column (cat. #10623). Injection volume is 1 μL (splitless). Injection temperature is 250° C. Maximum oven temperature is 220° C., with an initial setpoint temperature of 100° C. and ramp rate of 20° C./min. Carrier gas is helium. Flow rate is 1.8 mL/min. Pressure is 22.39 psi. A flame ionization detector at 220° C. is used.

HPLC is performed using an Agilent HPLC 1100 series with UV detector.

### Example 1

### 3,6-dimethylhexahydrobenzofuran-2(3H)-one Via H₂SO₄/H₂O₂/HOAc

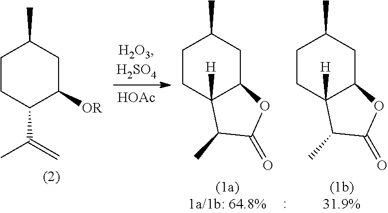

To a 500 ml 4-neck flask, equipped with a mechanical stirrer, is charged acetic acid 15 ml (0.26 mol, 2 eq.) and the flask is preheated to 90° C. Sulfuric acid (60% aqueous, 42 g, 0.26 mol, 2 eq.) and hydrogen peroxide (30% aqueous, 44.2 g, 3 eq.) are added via two separate dropping funnels. At the same time, isopulegol (20 g, 0.13 mol) is added by syringe pump at a rate of 40 ml/hr. The temperature is controlled at 90-95° C. during the addition process. The reaction mixture is further stirred at 95° C. for 2 hours, then it is cooled down in cold water and diluted with 100 ml of ice water. The mixture is extracted with EtOAc (3×50 ml). The combined EtOAc solutions are washed with water (30 ml), washed with saturated sodium carbonate (2×50 ml), washed with 10% thiodiglycol solution (30 ml), washed with brine (30 ml), and then dried over sodium sulfate, and concentrated to dryness under vacuum to obtain an oil. The oil is dissolved in 50 ml hexanes and passed through a plug of silica gel (50 g, 40 mm column) and eluted with EtOAc/hexane (8% to 12%). The eluate is concentrated to dryness to afford the crude product (13.5 g, 62% yield). GC analysis indicates a purity of 95.7%, and an isomeric ratio of isomer (1a) 64.8%; isomer (1b) 30.9% (isomer (1a) appears at about RT 11.87; isomer (1b) appears at about RT 12.49).

### Example 2

### Isomerization of 3,6-dimethylhexahydrobenzofuran-2(3H)-one

A crude mixture of 3,6-dimethylhexahydrobenzofuran-2(3H)-one obtained from a procedure analogous to Example 3, and having an isomeric ratio of 65:34 isomer (1a): isomer (1b), is dissolved in 70 mL heptane and the mixture is refluxed with a Dean-Stark apparatus to remove water until about 50 mL of heptane is collected. The reaction mixture is then cooled down to room temperature, and sodium tert-butoxide (0.2 g) is added. The mixture is stirred at room temperature for 18 hours and then another portion of sodium tert-butoxide (0.2 g) is added. The mixture is further stirred for 22 hours, and then it is diluted with water (30 ml) and extracted with methyl tert-butyl ether (MTBE) (30ml). The MTBE solution is washed with brine (20 ml), dried over sodium sulfate, and concentrated to dryness to afford 7.0 g oil. GC analysis shows that the isomeric ratio is 93.6% isomer (1a) to 6.4% isomer (1b).

### Example 3

### Crystallization of 3,6-dimethylhexahydrobenzofuran-2(3H)-one

A crude mixture of 3,6-dimethylhexahydrobenzofuran-2(3H)-one (52 g), containing 90.8% of product with the isomers (1a) and (1b) in a 94.8:5.2 ratio, is dissolved in 209 g n-hexane with stirring over ten minutes. The mixture is then placed in a freezer at −10° C. for 15 hours. A white crystalline product is observed to have formed. The crystals are separate by filtration and washed with 10 mL of cold n-hexane. The crystals are then transferred to a 50-mL round bottom flask and heated neat in an oil bath at 50° C. The resulting liquid is dried under high vacuum (about 5 Torr) at 50° C. for 2 hours with stirring. The liquid is then transferred to a flat surface, where it solidifies. The solid is broken up to form flakes. 29 g of solid flakes are collected. GC analysis shows the major isomer present at 98.6% (1a).

### Example 4

### 3,6-dimethylhexahydrobenzofuran-2(3H)-one Via Peracetic Acid (One-Pot Procedure)

To 2 g (0.013 mol) of isopulegol is added peracetic acid dropwise (˜ 3 ml from a total of 9.3 g, 0.039 mol, 3 eq.) over a period of 10 minutes at room temperature. An exothermic phenomenon is observed during the process. The reaction mixture is then stirred at ambient temperature for 1 hour. A mixture of triflic acid in acetic acid (1 g TfOH, 2 mL HOAc) is added dropwise. The reaction mixture is stirred at ambient temperature for 2 hours. GC analysis shows that the desired lactone is formed, and the major isomer content is 48.0%, and the minor isomer content is 24.8%. The total product yield by GC is 73%. The reaction may also be performed by combining peracetic acid, triflic acid, and acetic acid at the outset.

### Example 5

### 3,6-dimethylhexahydrobenzofuran-2(3H)-one

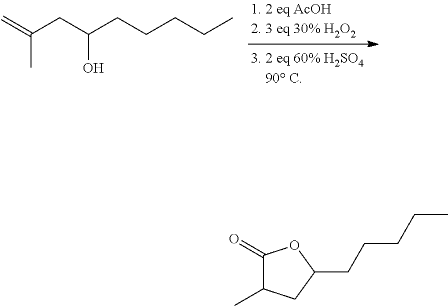

To a 40 ml reaction vial is added 0.37 ml of acetic acid and the solution is stirred at 90° C. for 5 minutes. In a sequence of one drop each, respectively, hydrogen peroxide (30% aq.), 2-methylnon-1-en-4-ol, and sulfuric acid (60% aq.) are added to the vial. In total, 0.99 mL of hydrogen peroxide, 500 mg of 2-methylnon-1-en-4-ol, and 628 mg of sulfuric acid are added. The reaction mixture is stirred for 1 hour and monitored by GC. Once GC shows that the starting material has been consumed, the reaction mixture is quenched by adding 2 mL of water and then 4 mL of MTBE. The organic part is collected and washed with saturated Na₂CO₃, dried over sodium sulfate, filtered, and concentrated to vacuo to afford 500 mg of a yellow crude product. The crude material is purified by flash chromatography (1:99-3:97 EtOAc: Hexanes) to afford the final product as a colorless liquid (100 mg, 18% yield). GC indicates a mixture of two isomers in a 1.4:1 ratio. ¹H NMR (CDCl₃, 500 MHz), δ 0.82-0.89 (m, 3H, —CH₃), 1.25-1.40 (m, 7H, —CH₂—, —CH₃), 1.41-1.74 (m, 4H, —CH—), 1.96-2.01 and 2.07-2.12 (m, 2H, —CH₂—), 2.44-2.49 and 2.62-2.70 (m, 1H, —CH—), 4.30-4.37 and 4.48-4.51 (m, 1H, —CHO—). Proton NMR confirms the identity of the product by comparison to literature precedent (see Sakai, K.; Oisaki K.; Kanai, M. Synthesis 52, 2171-2184).

### Example 6

### 3,6-dimethylhexahydrobenzofuran-2(3H)-one Via Peracetic Acid (Alternative One-Pot Procedure)

To a 500 ml 4-neck flask is charged 15 mL of acetic acid (0.26 mol) and the flask is preheated to 80° C. Sulfuric acid (60% aq., 64 g, 0.26 mol) and 15% peracetic acid solution (63.6 g, 0.125 mol) (15% peracetic acid, 22% hydrogen peroxide, 16% acetic acid, 46% water; total active oxygen 13.7%) are added via two separate dropping funnels. At the same time, isopulegol (30 g, 0.195 mol) is added by syringe pump at a rate of 40 ml/hr. The temperature is controlled at 80-85° C. during the addition process. The reaction mixture is further stirred at 80° C. for 1.5 hours, then it is cooled down in cold water and diluted with 60 ml of ice water. The mixture is extracted with hexanes (3×60 ml). The combined hexane solution is washed with water (30 ml), 20% NaHSO₃ (30 ml), saturated sodium carbonate (100 ml), brine (30 ml), and then the crude solution is passed through a plug of silica gel, and eluted with EtOAc/hexane (8% to 15%). The eluate is concentrated to dryness and dissolved in 80 ml of hexanes. The hexane solution is isomerized with sodium tert-butoxide (0.6 g) and crystalized as described in Example 4 and 5 to afford the product (7.8 g, 24%).

### Example 7

### 3,6-dimethylhexahydrobenzofuran-2(3H)-one Via Peracetic Acid (Two-Step Procedure)

To a 1 L 3-neck flask is charged isopulegol (100 g, 1.297 mol) and sodium acetate (8 g, 0.097 mol) and the mixture is cooled in ice-water bath. 32% Peracetic acid solution (216 g, 0.908 mol) (32% peracetic acid, 40-50% acetic acid, <8% hydrogen peroxide, balance water) is added dropwise over 40 minutes and then the reaction is further stirred in a water bath at about 20° C. for 1 hour.

In a 1-L 3-neck flask is charged 60% sulfuric acid (140 ml) and this is preheated to 60° C. Then the mixture from the first reaction step is added via syringe pump at 5 ml/min. At the same time, hydrogen peroxide (30% aqueous, 88 g) is added via syringe pump at 0.6 ml/min. The temperature is controlled at <62° C. during the additions. The reaction mixture is then further stirred at 60° C. for 1 hour after the hydrogen peroxide addition is finished. The reaction mixture is cooled down, diluted with water (100 ml), and extracted with heptane (200 ml, 2×150 ml). The combined heptane solutions are washed with water (100 ml), 10% Na₂SO₃, 2N NaOH (50 ml), brine (2×30 ml), and then dried (Na₂SO₄), and distilled under vacuum to afford the product (61.3 g, GC purity major isomer (1a) 60.1%, minor isomer (1b) 22.5%; GC yield 46%).

### Example 8

### 3,6-dimethylhexahydrobenzofuran-2(3H)-one Via Hydrogen Peroxide (Two-Step Procedure)

To a 1 L 3-neck flask is charged isopulegol (200 g, 1.297 mol) and sodium acetate (14g, 0.169 mol). The flask is preheated to 40° C. and then hydrogen peroxide (30% aqueous, 206 g, 1.816 mol) and acetic anhydride (211 g, 2.08 mol) are added separately via syringe pump at 4 ml/min. The temperature is controlled at 40-60° C. during the addition. After the addition is finished, the mixture is stirred at 45° C. for 1.5 hours, then the reaction mixture is cooled to about 15° C., and additional hydrogen peroxide is added (30% aqueous, 206 g, 1.816 mol).

In a 2-L Syrris Reactor is charged 100 g of acetic acid and 100 ml of sulfuric acid (60% aqueous, from total 530 g, 3.24 mol) and the mixture is preheated to 75° C. The reaction mixture from the first reaction step and the remaining sulfuric acid are added separately by dropping funnel over 85 minutes. The temperature is controlled at 80-88° C. during the additions. The reaction mixture is further stirred at 80° C. for 1.5 hours, then it is cooled down to below 20° C. The reaction mixture is diluted with water (400 ml), and extracted with heptane (500 ml, 2×250 ml). The combined heptane solutions are washed with water, 20% NaHSO₃, 2N NaOH, brine, and then dried (Na₂SO₄), and the crude solution is passed through a plug of silica gel and eluted with EtOAc/hexane (8% to 15%). The collected eluate is concentrated to dryness and dissolved in 650 ml of hexanes. To the hexane solution is added sodium tert-butoxide (5 g). The mixture is stirred overnight, and then it is quenched by addition of water (50 ml). The phases are separated, and the organic layer is washed with 0.5 N NaOH, brine, and dried (Na₂SO₄). recrystallized at −20° C., as described in Example 5. The obtained crystals are washed with cold hexane to afford the product (81 g, 37%).

### Example 9

### 3,6-dimethylhexahydrobenzofuran-2(3H)-one Via Hydrogen Peroxide (Two-Step Procedure With Co-Solvent)

To a 500 ml 3-neck flask is charged isopulegol (50 g, 0.324 mol) and sodium acetate (1.25 g). The flask is preheated to 40° C. in a water bath, and then hydrogen peroxide (30% aqueous, 50 ml from a total of 95.2 g, 0.843 mol) and acetic anhydride (52.9 g, 0.519 mol) are added separately via dropping funnels over 40 minutes. The temperature is controlled at 40-60° C. during the additions. The reaction is stirred in the water bath for 1 hour. The reaction mixture is then cooled to below 20° C. and the remaining hydrogen peroxide is added.

In a 1000 mL 3-neck flask is charged 10 ml acetic acid and 10 ml of sulfuric acid (60% aqueous, from a total of 106 g, 0.65 mol) and heptane (80 ml). The mixture is heated in a 90° C. heating bath and the reaction mixture from the first reaction step, and the rest of the sulfuric acid, are added separately over 50 minutes. The reaction mixture is gently refluxed during the process. The reaction mixture is further stirred for 1 hour after the additions. The reaction mixture is then was cooled down, diluted with water (100 ml), the phases are separated, and the aqueous layer is extracted with heptane (2×50). The combined heptane solutions are washed with water, 20% NaHSO₃, 2N NaOH, brine, dried (Na₂SO₄), and then the crude solution is passed through a plug of silica gel and eluted with EtOAc/hexane (8% to 15%). The eluate is concentrated to dryness. The crude product is dissolved in 250 ml of hexanes, isomerized using sodium tert-butoxide, and recrystallization from hexane, as described in Example 4 and 5, to afford the product (21.2g, 39%).

### Example 10

### 3,6-dimethylhexahydrobenzofuran-2(3H)-one Via Oxone

To a 2 L three-necked flask are added (−) isopulegol (26.8 g, 0.174 mol), acetone (160 mL, 2.160 mol) and ethyl acetate (270 mL), and the mixture is stirred vigorously. An aqueous solution of Oxone® (121.0 g, 0.398 mol; in water 500 mL) is added dropwise over 1 hour maintaining the temperature at 20 to 25° C. The reaction mixture is then stirred for an additional 7 hours. The organic layer is separated, the aqueous layer is extracted with ethyl acetate (300 mL), and the combined organic layers are washed with 20% (w/v) aqueous sodium chloride (250 mL) and then dried over Na₂SO₄ (50 g). The solution is evaporated to dryness, resulting in a gummy oil (26.0 g, 89.65% crude yield). The crude gummy oil is dissolved in n-hexane (250 mL) isomerized with sodium tert-butoxide (0.78 g), as described above, to afford the product (13.0 g, 33% yield; by GC isomer (1a) 69.50%, and isomer (1b) 4.84%.)

### Example 11

### 2-methyl-hexanoic Acid From 2-methyl-1-hexene (One-Pot, Two Step Procedure)

To a mixture of 2-methyl-1-hexene (7.1 g, 0.072 mol) and sodium acetate (0.6 g, 0.009 mol) is added hydrogen peroxide (30% aqueous, 11.5 g, 0.101 mol) and acetic anhydride (12.6 g, 0.123mol) dropwise over 50 min at 40° C. via separate dropping funnels. The reaction is then stirred overnight at room temperature.

Sulfuric acid (60% aqueous, 12 g) is heated in a separate flask to 70° C. The reaction mixture from the first step is then slowly added over 30 minutes, and the reaction is then refluxed for 1.5 hours, then cooled to 45° C. Additional hydrogen peroxide (30% aqueous, 6.6 g, 0.058 mol) is added over 30 minutes, and the reaction is stirred for 1.5 hours then cooled, diluted with water (50 ml), and the phases are separated. The organic layer is washed with water, 20% NaHSO₃, brine, and dried (Na₂SO₄) to afford the crude product (4.2 g, GC purity 60%, GC yield 26%).

The Examples provided herein are exemplary only and are not intended to be limiting in any way to the various aspects and embodiments of the invention described herein.

1. A method of making Compound of Formula I (Compound 1) comprising the step of treating a Compound of Formula II (Compound 2) with an oxidizing agent and an acid or base in a suitable solvent (e.g., aqueous solvent): 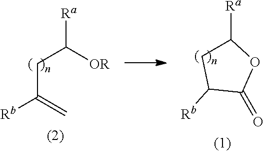
wherein the reaction proceeds in a single vessel without the isolation of any intermediates;
wherein R is H or a protecting group (e.g., an ether, an ester, or a silyl ether protecting group), R^{a} and R^{b} are each independently selected from H, optionally substituted C₁₋₆alkyl, or optionally substituted aryl, or wherein R^{a} and R^{b} together form a 4-10 membered optionally substituted cycloalkyl ring, and n is an integer from 1 to 5 (e.g., 1, 2 or 3).

2. The method according to claim 1, wherein R is H.

3. The method according to claim 1, wherein R is an ester protecting group, e.g., R is —C(O)—R¹, and wherein R¹ is H, C₁₋₆alkyl (e.g., methyl or ethyl), haloC₁₋₆alkyl (e.g., chloromethyl or trifluoromethyl), C₁₋₆alkoxy (e.g., methoxy or ethoxy), C₁₋₆alkoxymethyl (e.g., methoxyethyl or ethoxymethyl), aryl (e.g., phenyl), arylmethyl (e.g., benzyl), aryloxy (e.g., phenoxy), or aryloxymethyl (e.g., phenoxymethyl).

4. The method according to claim 3, wherein R is —C(O)—R¹, and wherein R¹ is methyl, ethyl, propyl, isopropyl, or tert-butyl.

5. The method according to claim 3, wherein R is —C(O)—R¹, and wherein R¹ is methyl.

6. The method according to claim 1, wherein the Compound (1) is Compound (1a), Compound (1b), Compound (1c), Compound (1d), or a mixture thereof:

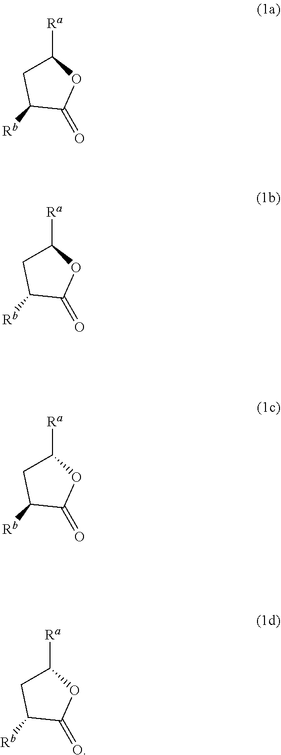

7. The method according to claim 1, wherein R^{a} and/or R^{b} is H.

8. The method according to claim 1, wherein R^{a} is optionally substituted C₁₋₆alkyl.

9. The method according to claim 1, wherein R^{a} is optionally substituted aryl.

10. The method according to claim 1, wherein R^{b} is optionally substituted C₁₋₆alkyl.

11. The method according to claim 1, wherein R^{b} is optionally substituted aryl.

12. The method according to claim 1, wherein R^{a} and R^{b} together form a 4-10 membered optionally substituted cycloalkyl ring (e.g., a cyclobutane, cyclopentane, or cyclohexane ring).

13. The method according to claim 1, wherein n is an integer selected from 1, 2, 3, 4 or 5.

14. The method according to claim 1, wherein n is an integer selected from 1 or 2.

15. The method according to claim 1, wherein the oxidizing agent is selected from one or more of hydrogen peroxide, a chromium oxidant (e.g., chromium trioxide, chromic acid, pyridinium chlorochromate, potassium dichromate, chromium trioxide-pyridine complex, pyridinium dichromate), osmium tetroxide, potassium permanganate, peracetic acid, perchloric acid, perbenzoic acid, meta-chloroperoxybenzoic acid (mCPBA), trifluoroperacetic acid, periodic acid, magnesium monoperoxyphthalate, dimethyl dioxirane (DMDO), tert-butyl hydroperoxide, sodium hypochlorite, sodium tungstate, sodium periodate, potassium periodate, iodosyl benzene, pentafluoroiodosyl benzene, cumene hydroperoxide, potassium persulfate, potassium monoperoxysulfate, pyridine N-oxide, 2,6-dichloropyridine N-oxide, sodium chlorite, sodium hypochlorite, sodium chlorate, sodium perchlorate, or oxygen (e.g., in combination with a transitional metal catalyst, e.g., an iron catalyst), optionally in combination with any secondary reagents (e.g., secondary oxidants, catalysts, complexing agents, directing agents, reducing agent, or chiral auxiliaries).

16. The method according to claim 15, wherein the oxidizing agent is hydrogen peroxide, peracetic acid, trifluoroperacetic acid, meta-chloroperoxybenzoic acid, or tert-butyl hydroperoxide.

17. The method according to claim 1, wherein the reaction comprises an acid, and the acid is a Bronsted acid, e.g., selected from hydrochloric acid, perchloric acid, sulfuric acid, phosphoric acid, acetic acid, trifluoromethanesulfonic acid, methanesulfonic acid, trifluoroacetic acid, and nitric acid, or a heteropoly acid (e.g., phosphotungstic acid).

18. The method according to claim 1, wherein the reaction is carried out in a solvent selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid), carbon dioxide (e.g., supercritical carbon dioxide), and carbon disulfide, or a combination thereof.

19. The method according to claim 18, wherein the solvent is acetic acid.

20. The method according to claim 1, wherein the reaction is carried out using a combination of hydrogen peroxide, sulfuric acid, and acetic acid, optionally 1.5-4 equivalents of each (e.g., 2-3 equivalents of each), or wherein the reaction is carried out using a combination of peracetic acid (e.g., 2-4 equiv. or about 3 equiv.), trifluoromethanesulfonic acid (e.g., 0.1-2 equiv., or about 0.5 equiv.), and acetic acid (e.g., 2-3 equiv., or about 2.5 equiv.).

21. The method according to claim 1, wherein the oxidizing agent is hydrogen peroxide, peracetic acid, trifluoroperacetic acid, meta-chloroperoxybenzoic acid, or tert-butyl hydroperoxide; and wherein the acid is selected from sulfuric acid, phosphoric acid, trifluoromethanesulfonic acid, and nitric acid; and wherein the solvent is acetic acid.

22. The method according to claim 1, wherein the method further comprises a Step (2) of treating the Compound (1) with a base to cause enrichment of the Compound (1a) by isomerization of the Compound (1b) to the Compound (1a).

23. Compound (1), wherein the compound is made according to the method of claim 1.

24. A product or composition, such as an organoleptic composition, comprising Compound (1), made according to claim 1.
